(19) United States
(12) Patent Application Publication
(10) Pub. No.: US 2019/0350643 A1
(43) Pub. Date: Nov. 21, 2019

(54) SURGICAL DEVICE

(71) Applicant: OLYMPUS CORPORATION, Tokyo (JP)

(72) Inventors: Reisuke OSADA, Hino-shi (JP); Ojiro KITAMURA, Hachioji-shi (JP)

(73) Assignee: OLYMPUS CORPORATION, Tokyo (JP)

(21) Appl. No.: 16/525,920

(22) Filed: Jul. 30, 2019

Related U.S. Application Data
(63) Continuation of application No. PCT/JP2017/004423, filed on Feb. 7, 2017.

(51) Int. Cl. A61B 18/14

(52) U.S. Cl. CPC A61B 18/1445; A61B 2017/00424

(57) ABSTRACT

A surgical device includes a housing, a rotator provided on the housing and rotatable about a first axis with respect to the housing, and an operator provided on the rotator and rotatable with respect to the rotator about a second axis intersecting the first axis. The operator includes a spherical band having a spherical outer surface. The spherical band includes an exposed portion which is exposed to the outside of the housing.

### CROSS-REFERENCE TO RELATED APPLICATIONS

This is a Continuation Application of PCT Application No. PCT/JP2017/004423, filed Feb. 7, 2017, the entire contents of which are incorporated herein by reference.

The present disclosure relates to a surgical device for treating a treatment target with an end effector.

### BACKGROUND

There are known surgical devices that can adjust an angle of an end effector about a longitudinal axis with respect to a housing. Such surgical devices may include an operating member to which an operation to bend the end effector with respect to a shaft is input. However, such operating members can be difficult to use and operability may vary based on a size of a user's hand.

### SUMMARY

According to one aspect of the present disclosure, a surgical device includes a housing, a rotator provided on the housing and rotatable about a first axis with respect to the housing, and an operator provided on the rotator and rotatable with respect to the rotator about a second axis intersecting the first axis. The operator includes a spherical band having a spherical outer surface. The spherical band includes an exposed portion which is exposed to the outside of the housing.

Advantages will be set forth in the description which follows, and in part will be obvious from the description, or may be learned by practice. The advantages may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate exemplary embodiments, and together with the general description given above and the detailed description of the embodiments given below, serve to explain the principles of the present disclosure.

FIG. 1 is a schematic view of a treatment instrument according to an exemplary embodiment, viewed from one side in a width direction of a housing.

FIG. 2 is a schematic view of the treatment instrument according to an exemplary embodiment, viewed from the side opposite to the side on which a grip is provided with respect to a longitudinal axis.

FIG. 3 is a schematic view of the treatment instrument according to an exemplary embodiment, viewed from the proximal side.

FIG. 4 is a perspective view schematically showing a dial according to an exemplary embodiment.

FIG. 5 is a schematic view of the dial according to an exemplary embodiment, viewed from the proximal side.

FIG. 6 is a schematic view of the dial according to an exemplary embodiment, viewed from one side in a direction along a rotation axis of the dial.

FIG. 7 is a perspective view schematically showing a region formed by rotation of the dial about the longitudinal axis, according to an exemplary embodiment.

FIG. 8 is a schematic view of a region formed by rotation of the dial about a longitudinal axis, viewed from the proximal side, according to an exemplary embodiment.

FIG. 9 is a schematic view of a state in which the housing is held by one hand, according to an exemplary embodiment, viewed from one side in the width direction of the housing.

FIG. 10 is a schematic view of a state in which the housing is held by one hand, according to an exemplary embodiment, viewed from the proximal side.

FIG. 11 is a perspective view schematically showing a region formed by rotation of a dial about the longitudinal axis, according to a comparative example.

FIG. 12 is a schematic view of a region formed by rotation of a dial about a longitudinal axis, according to the comparative example, viewed from the proximal side.

FIG. 13 is a schematic view of a state in which the housing is held by one hand when the dial is positioned at a first position about the longitudinal axis, according to the comparative example, viewed from the proximal side.

FIG. 14 is a schematic view of a state in which the housing is held by one hand when the dial is positioned at a second position different from the first position about the longitudinal axis, according to the comparative example, viewed from the proximal side.

FIG. 15 is a schematic view of a dial according to an exemplary embodiment, viewed from the proximal side.

FIG. 16 is a schematic view of a dial according to an exemplary embodiment, viewed from one side in a direction along a rotation axis of the dial.

### DETAILED DESCRIPTION

An exemplary embodiment will be described with reference to FIGS. 1 to 10. FIGS. 1 to 3 are views showing a treatment instrument 1 that is a surgical device of the present embodiment. As shown in FIGS. 1 to 3, the treatment instrument 1 has a longitudinal axis C. Here, one side in the direction along the longitudinal axis C is referred to as a distal side (arrow C1 side), and the side opposite to the distal side is referred to as a proximal side (arrow C2 side).

The treatment instrument 1 includes a holdable housing 2, a shaft (sheath) 3 connected to the distal side of the housing 2, and an end effector 5 provided at the distal portion of the shaft 3. The shaft 3 extends along the longitudinal axis C from the proximal side to the distal side. The shaft 3 is rotatable about the longitudinal axis C (first axis) with respect to the housing 2.

The housing 2 includes a housing body 11 extending along the longitudinal axis C, and a grip (fixed handle) 12 extending from the housing body 11 along a direction intersecting with the longitudinal axis C. The grip 12 is provided at a position away from the longitudinal axis C. One end of a cable 13 is connected to the grip 12. The other end of the cable 13 is connected to an energy control device (not shown).

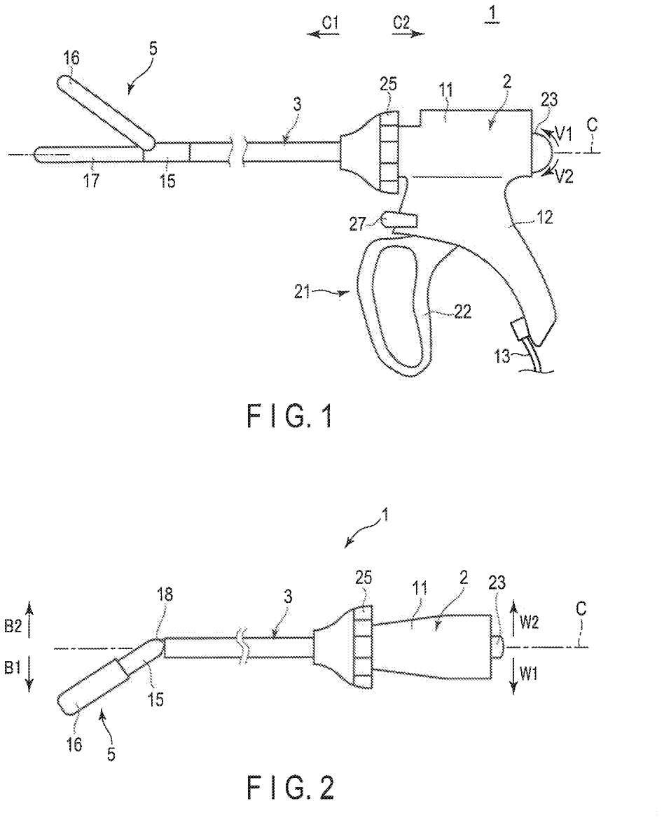

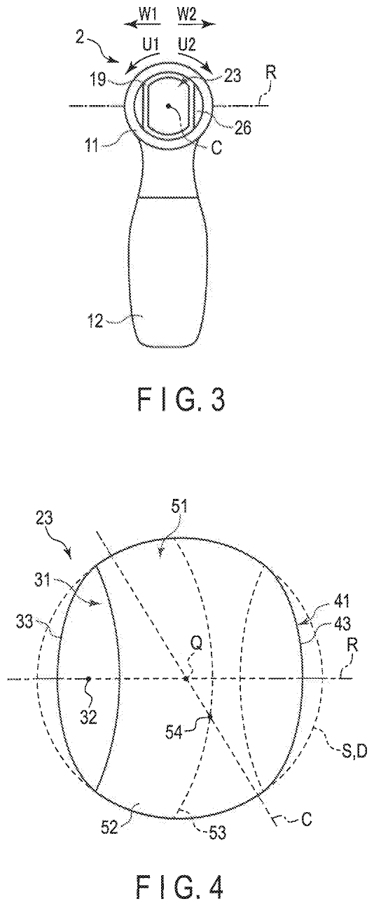

Here, the direction intersecting with (substantially perpendicular to) the longitudinal axis C and intersecting with (substantially perpendicular to) the extending direction of the grip 12 is set as the width direction of the housing 2 (directions indicated by an arrow W1 and an arrow W2). FIG. 1 is a view of the treatment instrument 1, viewed from one side (arrow W1 side) in the width direction of the housing 2. FIG. 2 is a view of the treatment instrument 1, viewed from the side opposite to the side on which the grip 12 is located with respect to the longitudinal axis C. FIG. 3 is a view of the treatment instrument 1, viewed from the proximal side.

The end effector 5 is rotatable about the longitudinal axis C with respect to the housing 2 together with the shaft 3 and is bendable with respect to the shaft 3 (longitudinal axis C). The angle (angular position) of the end effector 5 about the longitudinal axis C with respect to the housing 2 is changed by rotation of the end effector 5 about the longitudinal axis C with respect to the housing 2. In addition, the bending direction (directions indicated by the arrow B1 and the arrow B2) of the end effector 5 intersects with (is substantially perpendicular to) the longitudinal axis C. The end effector 5 includes a relay member 15, a first grasping piece 16, and a second grasping piece 17. The relay member 15 is bendably attached to the shaft 3 at the distal end of the shaft 3. That is, a bending joint 18 is formed between the shaft 3 and the relay member 15.

In addition, in the end effector 5, a gap between a pair of grasping pieces 16 and 17 can be opened and closed. The opening and closing direction of the grasping pieces 16 and 17 intersects with (is substantially perpendicular to) the longitudinal axis C and intersects with (is substantially perpendicular to) the bending direction of the end effector 5. The treatment target is grasped between the grasping pieces 16 and 17 by closing the grasping pieces 16 and 17 in a state in which the treatment target is disposed between the grasping pieces 16 and 17.

Here, in one embodiment, one of the grasping pieces 16 and 17 is integrated with or fixed to the shaft 3, and the other thereof is rotatably attached to the shaft 3. In addition, in another embodiment, both of the grasping pieces 16 and 17 are rotatably attached to the shaft 3. Furthermore, in another embodiment, a rod member (not shown) extends from the inside of the relay member 15 toward the distal side, and one of the grasping pieces 16 and 17 is formed by a projection portion of the rod member from the distal end of the relay member 15 to the distal side. The other of the grasping pieces 16 and 17 is rotatably attached to the shaft 3.

A handle (movable handle) 21 is rotatably attached to the housing 2. The handle 21 rotates with respect to the housing 2 such that the handle 21 is opened or closed with respect to the grip 12. That is, the handle 21 can be opened and closed with respect to the grip 12. In the handle 21, an operating force to open or close the handle 21 with respect to the grip 12 is applied. The operating force is applied in the handle 21 such that the handle 21 is opened or closed with respect to the grip 12. The handle 21 is located on the side on which the grip 12 is located with respect to the longitudinal axis C and is located on the distal side with respect to the grip 12. That is, the treatment instrument 1 of the present embodiment is a pistol type treatment instrument 1. In addition, the moving direction of the handle 21 with respect to the grip 12 in the opening and closing operation of the end effector 5 is substantially parallel to the longitudinal axis C. The movable member (not shown) extending inside the shaft 3 moves along the longitudinal axis C with respect to the shaft 3 and the housing 2 by opening or closing the handle 21 with respect to the grip 12. Therefore, at least one of the grasping pieces 16 and 17 rotates with respect to the relay member 15, and the grasping pieces 16 and 17 is opened or closed.

A rotation knob 25 that is a rotation operation input portion is attached to the distal side of the housing body 11. The shaft 3 is attached to the housing body 11 of the housing 2 in a state in which the shaft 3 is inserted into the inside of the housing body 11 from the distal side. In addition, the rotation knob 25 is connected to the shaft 3. The rotation knob 25 is rotatable about the longitudinal axis C with respect to the housing 2 together with the shaft 3 and the end effector 5. Therefore, the shaft 3 and the end effector 5 rotate about the longitudinal axis C with respect to the housing 2 together with the rotation knob 25 by rotating the rotation knob 25 about the longitudinal axis C with respect to the housing 2.

An operation button 27 is attached to the housing 2 as an operation input portion. In one embodiment, when the operation button 27 is pressed, an operation (an operation signal) for causing the treatment instrument 1 to output electrical energy is input to the energy control device. For example, as in a known treatment instrument, at least one of high frequency current, ultrasonic vibration, and heat is applied, as treatment energy, to the treatment target grasped between the grasping pieces 16 and 17. In one embodiment, an electric motor (not shown) may be driven by performing an operation input with the operation button 27, and a staple may be punctured on the treatment target grasped between the grasping pieces 16 and 17. Note that, Instead of or in addition to the operation button 27, a foot switch or the like separate from the treatment instrument 1 may be provided as the operation input portion.

A rotatable member (e.g., rotor, rotator) 26 extending along the longitudinal axis C is attached to the inside of the housing body 11. The rotatable member 26 is a rotatable body that is attached rotatably about the longitudinal axis C with respect to the housing body 11. The rotatable member 26 is connected to the rotation knob 25 and/or the shaft 3 inside the housing body 11. The rotatable member 26 rotates about the longitudinal axis C with respect to the housing 2 together with the rotation knob 25, the shaft 3, and the end effector 5 by rotating the rotation knob 25 about the longitudinal axis C with respect to the housing 2.

At the proximal end of the housing 2, the interior of the housing body 11 opens towards the proximal side. Therefore, at the proximal end of the housing body 11, an opening 19 is formed to open toward the outside. A bending operation dial (operator) 23 is attached to the rotatable member 26. The dial (operation member) 23 is a bending operation input portion to which an operation to bend the end effector 5 with respect to the shaft 3 is input. A part of the dial 23 is exposed to the outside of the housing 2 from the opening 19.

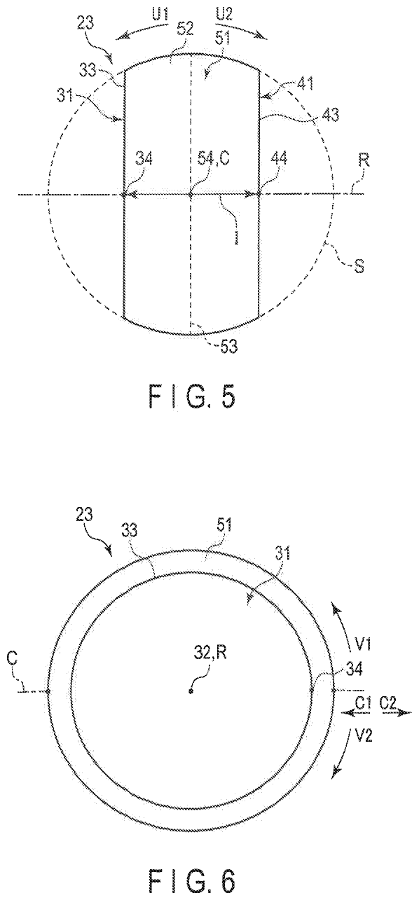

FIGS. 4 to 6 show the dial 23. FIG. 4 is a perspective view. FIG. 5 is a view viewed from the proximal side along the longitudinal axis C. The dial 23 has the rotation axis R (second axis). The rotation axis R intersects with (is substantially perpendicular to) the longitudinal axis C. FIG. 6 is a view viewed from one side in the direction along the rotation axis R. The dial 23 is rotatable about the rotation axis R (in the directions indicated by arrows V1 and V2) with respect to the rotatable member 26. The dial 23 is pivotally supported by the rotatable member 26 through, for example, a shaft member extending along the rotation axis R. When the dial 23 rotates about the rotation axis R, bending wires (not shown) extending in the inside of the housing 2 and the inside of the shaft 3 move along the longitudinal axis C with respect to the housing 2 and the shaft 3. Therefore, the end effector 5 bends with respect to the shaft 3.

The dial 23 rotates about the longitudinal axis C (in the directions indicated by arrows U1 and U2) with respect to the housing 2 together with the rotatable member 26. Therefore, when the rotation knob 25 rotates about the longitudinal axis C with respect to the housing 2, the dial 23 rotates about the longitudinal axis C with respect to the housing 2 together with the shaft 3, the end effector 5, and the rotatable member 26. Therefore, when the angle (angular position) of the rotation knob 25 rotates about the longitudinal axis C with respect to the housing 2, the angle (angular position) of the dial 23 about the longitudinal axis C with respect to the housing 2 also changes. In addition, when the dial 23 rotates about the longitudinal axis C with respect to the housing 2, the rotation axis R rotates about the longitudinal axis C together with the dial 23. Therefore, when the angle (angular position) about the longitudinal axis C of the dial 23 with respect to the housing 2 changes, the angle of the rotation axis R about the longitudinal axis C with respect to the housing 2 also changes. However, regardless of the angle of the rotation axis R about the rotation of the longitudinal axis C, the rotation axis R intersects with the direction along the longitudinal axis C.

The dial 23 includes a side surface 31 facing one side in the direction along the rotation axis R, a side surface 41 facing the side opposite to the side surface 31, and an operation surface 51 extending between the side surfaces 31 and 41. The side surfaces 31 and 41 are circular planes substantially perpendicular to the rotation axis R. The side surface 31 and the side surface 41 are planes having the same shape. The operation surface 51 is a curved surface extending around the rotation axis R and facing the outside of the dial 23 in the radial direction.

When the dial 23 is viewed from the direction along the rotation axis R, the side surface 31 has a circular shape with the center 32 as the center and the edge 33 of the side surface 31 as the circumference. That is, the side surface 31 is a first circular end surface provided on the dial 23 and having a circular shape when viewed from the direction along the rotation axis R. In addition, the side surface 41 is a plane having the same shape as the side surface 31. Therefore, the side surface 41 is a second circular end surface provided on the dial 23 and having a circular shape when viewed from the direction along the rotation axis R.

The operation surface 51 is a curved surface extending from the edge 33 of the side surface 31 to the edge 43 of the side surface 41 in the direction along the rotation axis R. Here, the dimension of the operation surface 51 in the direction along the rotation axis R is set as the width I of the operation surface 51. The width I of the operation surface 51 is a distance between the side surfaces 31 and 41 in the direction along the rotation axis R.

The longitudinal axis C passes through the central point Q of the operation surface 51 and the dial 23 in the direction along the rotation axis R. The central point Q is an intersection point between the rotation axis R and the longitudinal axis C. The operation surface 51 is formed in substantially the same shape as a part of the surface (spherical surface) of a virtual sphere S centered on the central point Q. The central point Q is a central point of the operation surface 51 and the virtual sphere S. Therefore, the distance from the central point Q is constant on the operation surface 51. In addition, the central point Q is located between the side surfaces 31 and 41 in the direction along the rotation axis R. Therefore, the operation surface 51 has a central position 53 located substantially at the same position as the central point Q in the direction along the rotation axis R.

A cross section substantially perpendicular to (intersecting with) the rotation axis R is circular on the operation surface 51. The diameter of the cross section substantially perpendicular to the rotation axis R increases as the operation surface 51 goes from the side surfaces 31 and 41 to the central position 53 in the direction along the rotation axis R. Therefore, the operation surface 51 is formed in a state of being separated from the rotation axis R as it goes from the side surfaces 31 and 41 to the central position 53 in the direction along the rotation axis R.

The dial 23 is formed in a shape sandwiched by the side surface 31 and the side surface 41 in the virtual sphere S when the virtual sphere S is cut by the side surface 31 and the side surface 41. That is, the dial 23 is a spherical base (a spherical segment) formed by a portion sandwiched between two planes when the sphere is cut by two virtual planes parallel to each other. The side surfaces 31 and 41 are the bottom surface of the spherical base formed by the dial 23. In addition, the operation surface 51 is a side surface of a spherical base formed by the dial 23. That is, the operation surface 51 is a spherical band formed by the surface of a portion (spherical base) sandwiched by two flat surfaces when the sphere is cut by two virtual planes parallel to each other.

In addition, the longitudinal axis C passes through the proximal end and the distal end of the operation surface 51. One of two intersection points between the operation surface 51 and the longitudinal axis C is located on the most proximal side in the operation surface 51 and the dial 23, and the other thereof is located on the most distal side in the operation surface 51 and the dial 23. Of the two intersection points between the operation surface 51 and the longitudinal axis C, a point located on the proximal side is referred to as an intersection point 54. The intersection point 54 is located on the central position 53 of the operation surface 51. In addition, the operation surface 51 is formed in a state of going to the distal side as it goes away from the intersection point 54.

In the dial 23 of the present embodiment, a part of the operation surface 51 and a part of the side surfaces 31 and 41 are exposed from the housing 2. The operation surface 51 has an exposed surface (exposed portion) 52 exposed from the opening 19 to the outside of the housing 2. The distal end of the exposed surface 52 of the operation surface 51 is located between the central point Q and the proximal end positions 34 and 44 of the side surfaces 31 and 41 in the direction along the longitudinal axis C. Note that, in the dial 23, only a part of the operation surface 51 may be exposed from the housing 2, and the side surfaces 31 and 41 may not be exposed. In this case, the distal end of the exposed surface 52 of the operation surface 51 is located closer to the proximal side than the proximal end positions 34 and 44 of the side surfaces 31 and 41.

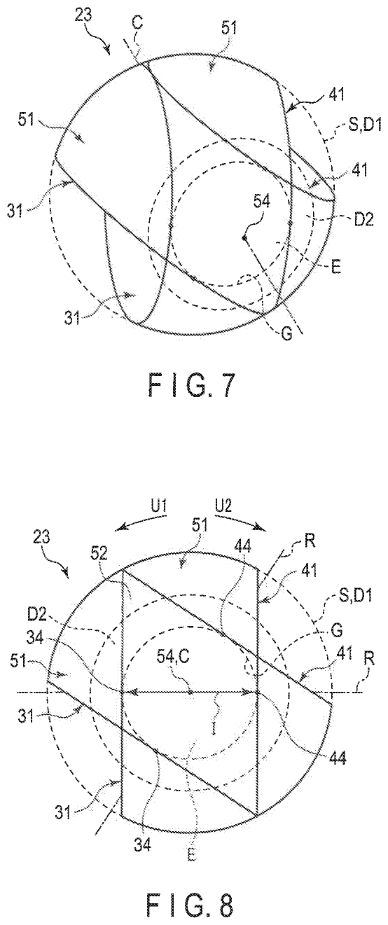

FIGS. 7 and 8 are views showing regions (trajectories) formed by the rotation of the dial 23 about the longitudinal axis C. FIG. 7 is a perspective view. FIG. 8 is a view viewed from the proximal side. A trajectory surface D1 is defined. The trajectory surface D1 is a region through which the operation surface 51 passes when the dial 23 rotates about the longitudinal axis C by 360° with respect to the housing 2. Here, the operation surface 51 has substantially the same shape as the surface of the virtual sphere S having the central point Q on the longitudinal axis C. Therefore, when the dial 23 rotates about the longitudinal axis C, the operation surface 51 rotates about the longitudinal axis C along the surface of the virtual sphere S. Therefore, the trajectory surface D1 has a virtual curved surface having substantially the same shape as the surface of the virtual sphere S.

In addition, a trajectory surface D2 is defined. The trajectory surface D2 is a region through which the exposed surface 52 of the operation surface 51 passes when the dial 23 rotates about the longitudinal axis C by 360° with respect to the housing 2. The trajectory surface D2 is a region formed on a part of the trajectory surface D1. The trajectory surface D2 is a virtual curved surface having a spherical crown shape formed when the virtual sphere S is cut by a virtual plane intersects with (substantially perpendicular) the longitudinal axis C.

In addition, a region in which the exposed surface 52 of the operation surface 51 exists regardless of the angle (angular position) of the dial 23 about the longitudinal axis C with respect to the housing 2 is set as a constant exist portion (common region) E. The constant exist portion (resident portion) E is a virtual region formed on a part of the trajectory surface D2 and the trajectory surface D1. In addition, in the constant exist portion E, even when the angle of the dial 23 about the longitudinal axis C with respect to the housing 2 changes, the position of the exposed surface 52 in the direction along the longitudinal axis C does not change.

When the dial 23 rotates about the longitudinal axis C, the exposed surface 52 rotates about the longitudinal axis C with the intersection point 54 as the center. Therefore, the intersection point 54 is located at a constant position, regardless of the angular position of the dial 23. Therefore, the constant exist portion E includes the intersection point 54. The intersection point 54 is located at the proximal end of the constant exist portion E. At any angle of the dial 23 about the longitudinal axis C with respect to the housing 2, the constant exist portion E is located on the exposed surface 52 that is a spherical band.

In addition, in the portion located closer to the proximal side than the proximal end positions 34 and 44 of the side surfaces 31 and 41 in the exposed surface 52 of the operation surface 51, the exposed surface 52 of the operation surface 51 exists regardless of the angle (angular position) of the dial 23 about the longitudinal axis C with respect to the housing 2. Therefore, the constant exist portion E is formed in the portion of the operation surface 51 from the proximal end positions 34 and 44 of the side surfaces 31 and 41 to the intersection point 54 in the direction along the longitudinal axis C. Therefore, the constant exist portion E is a virtual curved surface having a spherical crown shape formed by a surface of a portion (spherical portion) formed when the dial 23 (or virtual sphere S) is cut by a virtual plane G which is substantially perpendicular to (intersects with) the longitudinal axis C. The virtual plane G is a circular plane located at the distal end of the constant exist portion E. The edge of the virtual plane G is formed by the trajectory of the exposed surface 52 at the proximal end positions 34 and 44. The diameter of the plane G coincides with the width I of the operation surface 51.

The constant exist portion E is a part of the exposed surface 52 of the operation surface 51 and a part of the surface of the virtual sphere S. Therefore, in the constant exist portion E, the cross-sectional shape substantially perpendicular to (intersecting with) the longitudinal axis C is circular. In the constant exist portion E, the diameter of the cross section substantially perpendicular to the longitudinal axis C decreases from the distal side to the proximal side in the direction along the longitudinal axis C. Therefore, the constant exist portion E is formed to approach the longitudinal axis C as it goes from the distal side to the proximal side in the direction along the longitudinal axis C. The constant exist portion E is formed in a state of going to the distal side as it goes away from the intersection point 54. In addition, the constant exist portion E is formed at a position including a predetermined position T described later on the operation surface 51.

Next, the operation and effect of the treatment instrument 1 that is the surgical device of the present embodiment will be described. When the treatment target such as a body tissue is treated by using the treatment instrument 1, the operator holds the housing 2 with one hand (right hand or left hand) and inserts the end effector 5 into a body cavity such as an abdominal cavity. The handle 21 is closed with respect to the grip 12 in a state in which the treatment target is disposed between the grasping pieces 16 and 17. Therefore, the treatment target is grasped between the grasping pieces 16 and 17. The above-described treatment energy is applied to the treatment target grasped by performing an operation input with the operation button 27 in a state in which the treatment target is grasped.

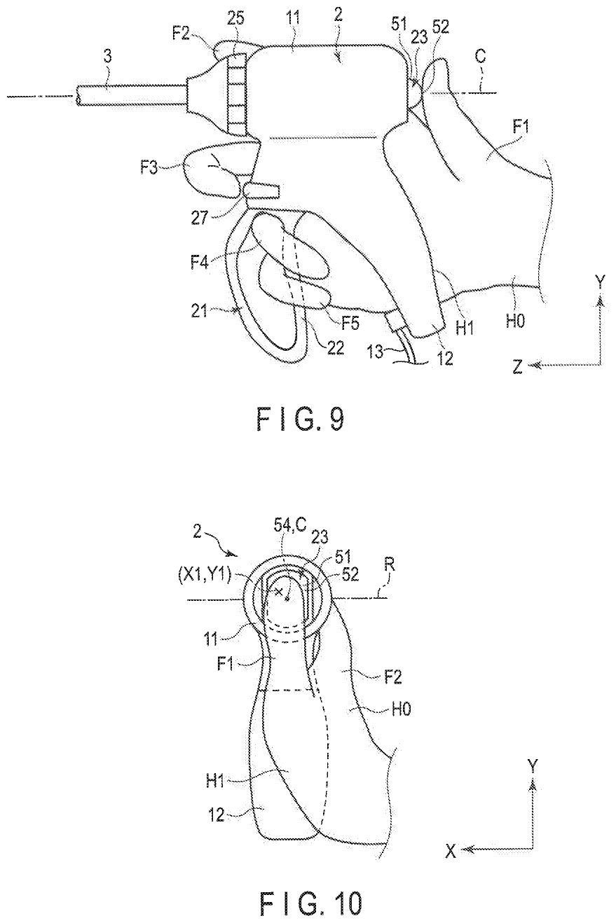

FIGS. 9 and 10 are views showing a state in which the housing 2 of the treatment instrument 1 is held by one hand (right hand in FIGS. 9 and 10) H0. FIG. 9 is a view seen from one side in the width direction of the housing 2, and FIG. 10 is a view viewed from the proximal side. As shown in FIGS. 9 and 10, in the pistol type treatment instrument such as the treatment instrument 1, the operator holds the housing 2 in a predetermined posture by using one hand (here, the right hand) H0. In a state in which the housing 2 is held in the predetermined posture, a palm H1 and a thumb F1 abut on the grip 12 from the proximal side. A ring finger F4 and a little finger F5 extend from the palm H1 toward the distal side and are hung on the handle 21. An operating force for opening or closing the handle 21 with respect to the grip 12 is applied to the handle 21 by the ring finger F4 and/or the little finger F5. A middle finger F3 extends from the palm H1 to the distal side toward an installation surface 28, and the belly of the middle finger F3 abuts on the installation surface 28 from the distal side. When the operation button 27 is pressed by the middle finger F3, the operation at the operation button 27 is input. An index finger F2 extends from the palm H1 to the distal side toward the rotation knob 25 and the belly of the index finger F2 abuts on the rotation knob 25. The operation input for rotating the end effector 5 about the longitudinal axis C with respect to the housing 2 is performed by rotating the rotation knob 25 by using the index finger F2. Note that at least one of the middle finger F3 and the index finger F2 may be hung on the handle 21, depending on the operator. In addition, the operation of the operation button 27 may be performed by the index finger F2, and the rotation operation by the rotation knob 25 may be performed by the middle finger F3.

The thumb F1 extends from the palm H1 toward the housing body 11 along the outer surface of the grip 12, and the belly of the thumb F1 abuts on the exposed surface 52 of the operation surface 51 of the dial 23 from the proximal side. The operation to bend the end effector 5 with respect to the shaft 3 is input by rotating the dial 23 by using the thumb F1.

Here, as shown in FIGS. 9 and 10, X, Y, and Z coordinates are defined. The X axis is an axis substantially parallel to the width direction of the housing 2. The Y axis is an axis intersecting with the longitudinal axis C and the width direction (X axis) of the housing 2. The Z axis is an axis substantially parallel to the longitudinal axis C and intersects with the X axis and the Y axis. In a state in which the housing 2 is held by one hand (here, the right hand) H0, the position (X coordinates) of the thumb F1 in the width direction is fixed. In a state in which the housing 2 is held by one hand (here, the right hand) H0, the thumb F1 is disposed at, for example, coordinates (X1, Y1) located closer to the upper left side than the intersection point 54, when viewed from the proximal side along the longitudinal axis C.

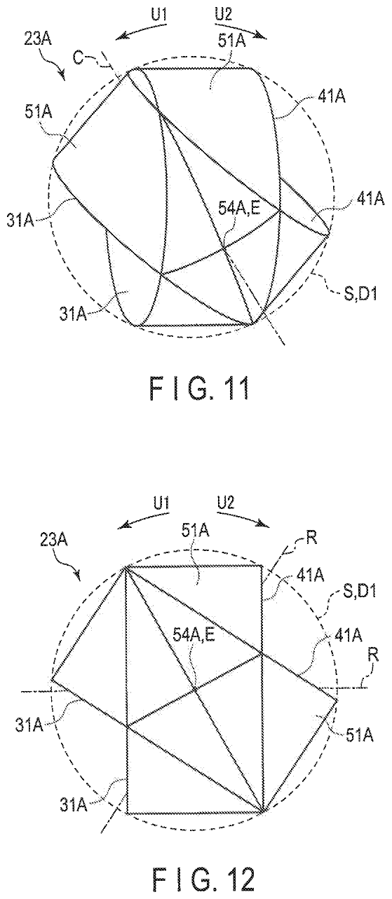

Next, a comparative example will be described with reference to FIGS. 11 and 12. FIGS. 11 and 12 show a region (trajectory) formed when the dial 23A according to the present comparative example rotates about the longitudinal axis C. FIG. 11 is a perspective view, and FIG. 12 is a view viewed from the proximal side. A dial 23A is formed in a cylindrical shape extending along the rotation axis R. The side surfaces 31A and 41A are the cylindrical bottom surface of the dial 23A, and the operation surface 51A is the cylindrical side surface of the dial 23A. In the dial 23A and the operation surface 51A, the cross section intersecting with (substantially perpendicular to) the rotation axis R is the same throughout the direction along the rotation axis R. In the operation surface 51A, even when the position changes in the direction along the rotation axis R, the distance from the rotation axis R is constant. In addition, the operation surface 51A is formed so as to be directed to the distal side as it goes away from the intersection point 54A with the longitudinal axis C around the rotation axis R.

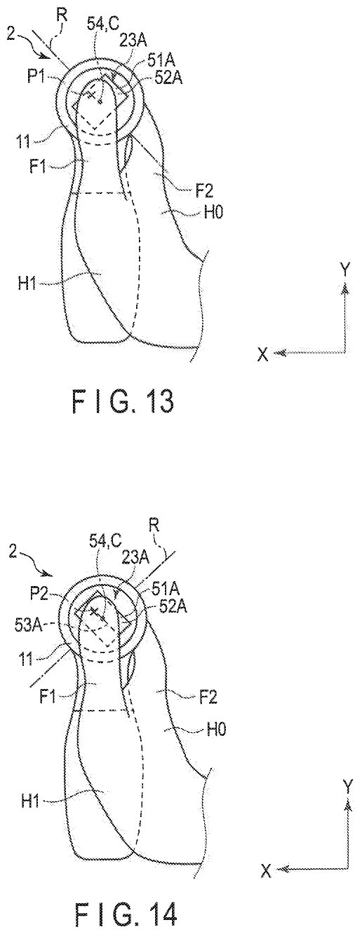

FIG. 13 is a view of a state (first state) in which the dial 23A rotates at an angle with respect to the housing 2, viewed from the proximal side along the longitudinal axis C. In this state, the rotation axis R viewed from the proximal side along the longitudinal axis C can be viewed as passing through the vicinity of the coordinates (X1, Y1). In addition, the rotation axis R when viewed from the proximal side along the longitudinal axis C extends obliquely with respect to the width direction of the housing 2, intersects with the intersection point 54A from the region closer to the upper left region than the intersection point 54A, and extends toward the region closer to the lower right region than the intersection point 54A.

The thumb F1 disposed at the coordinates (X1, Y1) abuts on the exposed surface 52A of the operation surface 51A at a first abutting position P1(X1, Y1, Z1) separated from the intersection point 54A along the rotation axis R. Here, even when the position changes in the direction along the rotation axis R, the distance from the rotation axis R is constant on the operation surface 51A. Therefore, the distance from the rotation axis R at the abutting position P1 is equal to the distance from the rotation axis R at the intersection point 54A. That is, the thumb F1 abuts on the operation surface 51A at the same position as the intersection point 54A in the direction (Z coordinate) along the longitudinal axis C.

FIG. 14 is a view showing a state (second state) in which the dial 23A rotates about the longitudinal axis C by 90° from the state shown in FIG. 13. In this state, the central position 53A of the operation surface 51A in the direction along the rotation axis R when viewed from the proximal side along the longitudinal axis C can be viewed as passing through the vicinity of the coordinates (X1, Y1). In addition, the rotation axis R when viewed from the proximal side along the longitudinal axis C extends obliquely with respect to the width direction of the housing 2, intersects with the intersection point 54A from the region closer to the lower left region than the intersection point 54A, and extends toward the region closer to the upper right region than the intersection point 54A.

In the second state, the abutting position P1(X1, Y1, Z1) is separated along the direction (about the rotation axis R) intersecting with the rotation axis R from the intersection point 54A. Here, the operation surface 51A is formed so as to be directed to the distal side as it goes away from the intersection point 54A about the rotation axis R. Therefore, at the coordinates (X1, Y1), the operation surface 51A is located closer to the distal side than the intersection point 54A. Therefore, at the coordinates (X1, Y1), the operation surface 51A is located closer to the distal side than the abutting position P1(X1, Y1, Z1).

Therefore, in order to operate the dial 23A in the second state shown in FIG. 14, it is necessary that the thumb F1 abuts the operation surface 51A by moving the thumb F1 to the distal side from a state in which the thumb F1 is placed at the abutting position P1(X1, Y1, Z1) in the first state shown in FIG. 13. At this time, the thumb F1 moves to the distal side and also moves downward in

FIGS. 13 and 14 in the direction intersecting with (substantially perpendicular to) the width direction of the housing 2. Therefore, in the second state, the thumb F1 abuts on the operation surface 51A at a second abutting position P2(X1, Y2, Z2) located closer to the distal side and lower than the first abutting position P1(X1, Y1, Z1).

As described above, in the present comparative example, the extending state of the operation surface 51A changes in the direction along the rotation axis R and the direction intersecting with the rotation axis R. Therefore, the position at which the thumb F1 abuts on the operation surface 51A changes in the direction (Z coordinate) along the longitudinal axis C and the direction (Y coordinate) intersecting with the longitudinal axis C and the width direction of the housing 2, according to the angle (angular position) of the dial 23A with respect to the housing 2. Therefore, the operator needs to move the thumb F1 according to the angle (angular position) of the dial 23A with respect to the housing 2.

In addition, the position at which the thumb F1 is disposed on the exposed surface 52A of the operation surface 51A may change according to the size of the operator's hand. In addition, as the position at which the thumb F1 is disposed is farther from the intersection point 54A, the distance for moving the position of the thumb F1 corresponding to the angle of the dial 23A with respect to the housing 2 is increased. Therefore, the difference in the size of the operator's hand may affect the operability.

In addition, when the dial 23A rotates about the longitudinal axis C with respect to the housing 2, the exposed surface 52A of the operation surface 51A rotates about the longitudinal axis C and the intersection point 54A with the longitudinal axis C. Therefore, the constant exist portion E includes the intersection point 54A. In addition, at positions other than the intersection point 54A on the exposed surface 52A when viewed from the proximal side along the longitudinal axis C, the position at which the thumb F1 abuts on the exposed surface 52A of the operation surface 51A changes according to the angle (angular position) of the dial 23A with respect to the housing 2. Therefore, in the present comparative example, the constant exist portion E is a point formed only by the intersection point 54A. Therefore, at positions other than the intersection point 54A on the exposed surface 52A, the feeling given to the operator's finger by the exposed surface 52A of the operation surface 51A changes according to the angle (angular position) of the dial 23A about the longitudinal axis C with respect to the housing 2.

On the other hand, in the present embodiment, the operation surface 51 is formed in a spherical band shape. Therefore, regardless of the angle (angular position) of the dial 23 about the longitudinal axis C with respect to the housing 2, the operation surface 51 is formed so as to be directed to the distal side as it goes away from the intersection point 54. Therefore, in the operation surface 51, even when the position changes in the direction along the rotation axis R, the operation surface 51 is formed so as to be directed to the distal side as it goes away from the intersection point 54, as in the case where the position changes about the rotation axis R.

In the present embodiment, the extending state of the operation surface 51 does not change in the direction along the rotation axis R and the direction intersecting with the rotation axis R. Therefore, for example, in the first state in which the rotation axis R passes through the vicinity of the coordinates (X1, Y1), the operation surface 51 is located closer to the distal side than the intersection point 54 at the coordinates (X1, Y1), as in the second state. Therefore, at the coordinates (X1, Y1), the operation surface 51 is located closer to the distal side than the abutting position P1(X1, Y1, Z1). Therefore, even in the first state, the thumb F1 abuts on the operation surface 51 at the second abutting position P2(X1, Y2, Z2) on the distal side and the lower side with respect to the intersection point 54, as in the second state.

As described above, in the present embodiment, the position at which the thumb F1 abuts on the operation surface 51 does not change regardless of the angle (angular position) of the dial 23 with respect to the housing 2. Therefore, the position at which the thumb F1 abuts on the operation surface 51 does not change even when the angle (angular position) of the dial 23 with respect to the housing 2 changes. Therefore, even when the angle (angular position) of the dial 23 with respect to the housing 2 changes, the operator can operate the dial 23 without moving the position of the thumb F1 to abut on the operation surface 51. That is, even when the angle (angular position) of the dial 23 with respect to the housing 2 changes, the operator can operate the dial 23 with the thumb F1 maintained at a predetermined position. Therefore, the operability at the dial 23 is improved.

In addition, in the present embodiment, the dial 23 can be operated without moving the position of the thumb F1 corresponding to the angle of the dial 23 with respect to the housing 2, regardless of the position at which the thumb F1 is disposed along the longitudinal axis C when viewed from the proximal side. Therefore, the influence of the size of the operator's hand on the operability is reduced.

In addition, in the present embodiment, the constant exist portion E is a virtual curved surface having a spherical crown shape. In a state in which the housing 2 is held, the operator's finger (thumb F1) is disposed inside the constant exist portion E. That is, a predetermined position of the thumb F1 is disposed inside the constant exist portion E. Therefore, the thumb F1 abuts on the exposed surface 52 of the operation surface 51 at the constant exist portion E. As described above, the exposed surface 52 of the operation surface 51 exists in the constant exist portion E, regardless of the angle of the dial 23 about the longitudinal axis C with respect to the housing 2, that is, at any angle of the dial 23 about the longitudinal axis C with respect to the housing 2. Therefore, the feeling given to the thumb F1 becomes constant, regardless of the angle of the dial 23 with respect to the housing 2. Therefore, the operator can operate the dial 23 with a certain sense, regardless of the angle (angular position) of the dial 23 about the longitudinal axis C with respect to the housing 2.

In addition, the width I of the operation surface 51 of the dial 23 is preferably formed as large as possible within the range that can be attached to the housing 2. When the width I of the operation surface 51 is formed to be large, the region in which the constant exist portion E is formed becomes large. In addition, the dial 23 is also preferably formed in a spherical shape. In this case, the dial 23 has substantially the same shape as the virtual sphere S, and the entire surface of the dial 23 is the operation surface 51. In this case, when the dial 23 rotates about the longitudinal axis C by 360°, the trajectory surface D2 through which the exposed surface 52 of the operation surface 51 passes is a virtual curved surface having a spherical crown shape formed when a sphere is cut by a virtual plane intersecting with (substantially perpendicular to) the longitudinal axis C. In addition, the constant exist portion E is a virtual curved surface having a spherical crown shape formed when the sphere cut by a virtual plane intersecting with (substantially perpendicular to) the longitudinal axis C.

Note that, in the present embodiment, the side surfaces 31 and 41 are planes substantially perpendicular to the rotation axis R, but the present disclosure is not limited thereto. For example, the side surfaces 31 and 41 may be curved surfaces. The side surfaces 31 and 41 may be, for example, curved surfaces in which the central portion protrudes toward the side away from the longitudinal axis C, and may be curved surfaces in which the central portion is recessed toward the longitudinal axis C. Even in these cases, the side surfaces 31 and 41 are circular end surfaces having a circular shape when viewed from the direction along the rotation axis R.

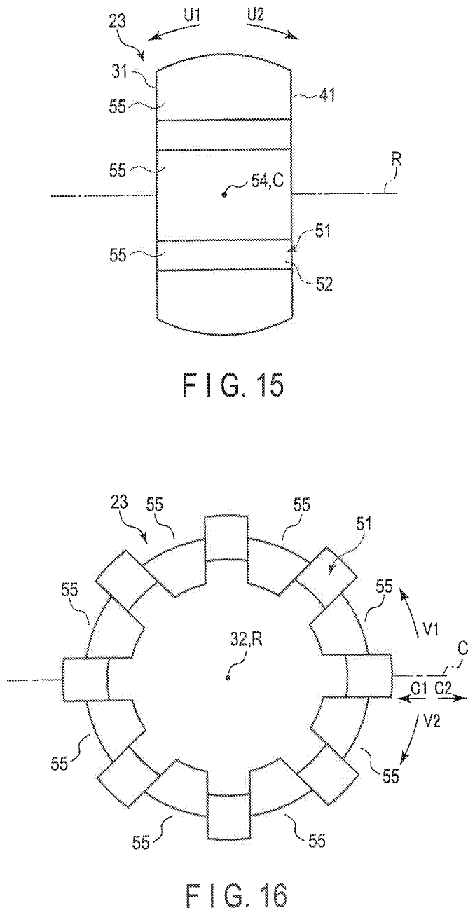

FIGS. 15 and 16 are views showing the dial 23 according to an exemplary embodiment. FIG. 15 is a view of the dial 23, viewed from the proximal side. FIG. 16 is a view of the dial 23, viewed from one side in the direction along the rotation axis R. As shown in FIGS. 15 and 16, a plurality of grooves 55 are provided on the operation surface 51 of the dial 23 as anti-slip members. In the grooves 55, the operation surface 51 is recessed toward the central point. The groove 55 extends along the operation surface 51 substantially in parallel with the rotation axis R.

In the present embodiment, when the dial 23 rotates about the longitudinal axis C by 360° with respect to the housing 2, the trajectory surface D2 through which the exposed surface 52 of the operation surface 51 passes has a virtual curved surface having a spherical crown shape formed when a virtual sphere S is cut by a plane substantially perpendicular to the longitudinal axis C, or a spherical band shape formed when a virtual sphere S is cut by two planes substantially perpendicular to the longitudinal axis C.

The groove 55 may extend in the direction about the rotation axis R along the central position 53 of the operation surface 51. In this case, the trajectory surface D2 has a virtual curved surface having a spherical band shape formed when the virtual sphere S is cut by two planes intersects with (is substantially perpendicular to) the longitudinal axis C.

In addition, instead of providing the grooves as the non-slip member, the operation surface 51 may be made of a material having a large coefficient of friction, such as a rubber.

In the above-described embodiments and the like, the surgical device (1) includes a housing (2), a rotatable body (26) provided on the housing (2) and rotatable about a first axis (C) with respect to the housing (2), and an operator (23) provided on the rotatable body (26) and rotatable about a second axis (R) intersecting with the first axis (C) with respect to the rotating body (26), wherein the operator (23) includes a spherical band (51) cut by two virtual planes (31, 41) whose spherical surfaces intersect with the second axis (R), and the spherical band (51) has an exposed portion (52) which is exposed to the outside of the housing (2).

Additional advantages and modifications will readily occur to those skilled in the art. Therefore, the present disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A surgical device comprising:
a housing;
a rotator provided on the housing and rotatable about a first axis with respect to the housing; and
an operator provided on the rotator and rotatable with respect to the rotator about a second axis intersecting the first axis, the operator including:
a spherical band that: (i) has a shape of a spherical segment, and (ii) includes an exposed portion exposed to an outside of the housing.

2. The surgical device according to claim 1, wherein:
the spherical band has an outer surface that extends between a first circular end surface facing in a direction along the second axis, and a second circular end surface facing in an opposite direction to the first circular end surface, and
the second axis passes through a center of the first circular end surface and a center of the second circular end surface.

3. The surgical device according to claim 1, wherein a trajectory shape of the exposed portion when the rotator rotates about the first axis with respect to the housing is a spherical cap shape or a spherical segment shape.

4. The surgical device according to claim 3, wherein the trajectory shape includes a common region at which a portion of the spherical band is always present regardless of an angular position of the operator about the first axis with respect to the housing.

5. The surgical device according to claim 4, wherein:
the housing includes a housing body extending from a proximal side to a distal side along the first axis, and a grip extending from the housing body along a direction intersecting the first axis,
the operator is provided at a proximal end of the housing body, and
the operator is configured to be actuated by a thumb of a user such that a belly of the thumb of the user is located at a predetermined position in the common region in a state in which the housing is held by a hand of the user in a predetermined posture.

6. The surgical device according to claim 4, wherein the common region has a spherical cap shape.

7. The surgical device according to claim 1, wherein the operator is configured to be actuated by a thumb of a hand of a user holding the housing.

8. The surgical device according to claim 1, wherein the operator is further rotatable around the first axis with the rotator.

9. The surgical device according to claim 1, wherein the spherical band includes a spherical outer surface formed such that a diameter of the band decreases from a central position of the spherical outer surface along the second axis toward an edge of the spherical band.

10. The surgical device according to claim 1, wherein the spherical band has a shape of a virtual sphere centered around the second axis that has been cut along two parallel planes intersecting the second axis.

11. The surgical device according to claim 1, wherein:
the housing includes a housing body extending from a proximal side to a distal side along the first axis;
the operator is provided at a proximal end of the housing body; and
a central point of an outer surface of the exposed portion along the second axis forms the proximal-most point of the spherical band.

12. The surgical device according to claim 1, wherein the first axis intersects the spherical band at a central point of an outer surface of the exposed portion of the spherical band along the second axis.

13. The surgical device according to claim 1, wherein a plurality of grooves are provided on an outer surface of the spherical band.

14. A surgical device comprising:
an end effector configured to treat a body tissue;
a sheath extending along a first axis from a proximal end to a distal end, the end effector being provided at the distal end of the sheath;
a housing that holds the proximal end of the sheath; and
an operator that is: (i) provided in the housing, (ii) rotatable about the first axis with respect to the housing, and (iii) configured to rotate with respect to the housing about a second axis intersecting the first axis, the operator including an operation surface having a spherical band shape with an intersection point between the first axis and the second axis as a central point.

15. The surgical device according to claim 14, wherein:
the first axis intersects the operator at:
a first intersection point that passes through a proximal-most point of the operator, and
a second intersection point that passes through a distal-most point of the operator.

16. The surgical device according to claim 14, wherein:
the operator is provided at a proximal end side of the housing along the first axis, and
a part of the operation surface is exposed from the housing.

17. The surgical device according to claim 14, wherein the operation surface includes a central position located substantially at the same position as the central point in a direction along the second axis.

18. The surgical device according to claim 17, wherein:
the operation surface extends between a first circular end surface facing in a direction along the second axis, and a second circular end surface facing in an opposite direction to the first circular end surface, and
a diameter of the operation surface in a cross section perpendicular to the second axis increases from an edge of the first circular end surface and an edge of the second circular end surface toward the central position in the direction along the second axis.
